(19) United States
(12) Patent Application Publication
(10) Pub. No.: US 2025/0195398 A1
(43) Pub. Date: Jun. 19, 2025

(54) COSMETIC COMPOSITION WITH IMPROVED SOLVENT SYSTEMS

(71) Applicant: L’ORÉAL, Paris (FR)

(72) Inventors: Xuefei Liu, Edison, NJ (US); Hy Si BUI, Piscataway, NJ (US); Ritesh Kumar SINHA, Linden, NJ (US)

(73) Assignee: L’ORÉAL, Paris (FR)

(21) Appl. No.: 18/543,619

(22) Filed: Dec. 18, 2023

(51) Int. Cl. A61K 8/92; A61K 8/37; A61Q 1/14

(52) U.S. Cl. CPC A61K 8/922; A61K 8/375; A61Q 1/14; A61K 2800/43

(57) ABSTRACT

A method for solubilizing a reaction product may be provided. The method may include providing a reaction product of an acrylate and a natural or food-derived oil, as well as providing one or more solvents for the reaction product. Each solvent may have Hansen Solubility Parameters (hydrogen bonding component (D), polar component (P), and dispersion component (H)) that satisfy

$$Ra \leq \sqrt{4 {( D - {D}_{1} )}^{2} + {( P - {P}_{1} )}^{2} + {( H - {H}_{1} )}^{2}} ,$$

where Ra=13.4 MPa^{0.5}, D₁=16.8 MPa^{0.5}, P₁=4.8 MPa^{0.5}, and H₁=13.0 MPa^{0.5}. A premix composition may therefore consist of the reaction product and the one or more solvents. A cosmetic composition may include the premix composition and one or more additional materials.

### TECHNICAL FIELD

The present application is drawn to cosmetic compositions, and specifically to such compositions with improved processibility.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Cosmetic chemists often experiment with new materials, or materials not typically utilized in the cosmetic industry. One such material is a reaction product of an acrylate and a natural or food-derived oil (such as linseed oil). However, such a material can be difficult to incorporate due to a high viscosity. As such, cosmetic grade solvents are identified for ease of processing to incorporate the reaction product into various cosmetic formulations.

### BRIEF SUMMARY

Various deficiencies in the prior art are addressed below by the disclosed compositions of matter and techniques.

In various aspects, a cosmetic premix composition may be provided. The cosmetic makeup composition may include a reaction product of a natural or food-derived oil and a methacrylate or acrylate polymer.

The cosmetic premix composition may include a solvent for the reaction product, the solvent having Hansen Solubility Parameters within a designated range, where the distance (Ra) between Hansen parameters in Hansen space must satisfy particular requirements. Specifically, the parameters must satisfy

$$Ra \leq \sqrt{4 {( D - {D}_{1} )}^{2} + {( P - {P}_{1} )}^{2} + {( H - {H}_{1} )}^{2}} ,$$

where Ra=13.4 MPa^{0.5}, D₁=16.8 MPa^{0.5}, P₁=4.8 MPa^{0.5}, and H₁=13.0 MPa^{0.5}, and the hydrogen bonding component (D), the polar component (P), and the dispersion component (H) may be defined by the selected solvent with units of MPa^{0.5}.

The one or more solvents may include, e.g., caprylic/capric triglyceride, isopropyl myristate, citronellyl acetate, acetone, triglycerides, or a combination thereof. In some embodiments, at least some of the triglycerides may be provided by a vegetable oil. The reaction product may be present in an amount of 10-80% by weight of the cosmetic premix composition. The natural or food-derived oil may be linseed oil.

In various aspects, a method of manufacturing a cosmetic composition may be provided. The method may include providing a reaction product of an acrylate and a natural or food-derived oil. The method may include providing one or more solvents. Each solvent may have Hansen Solubility Parameters that satisfy

$$Ra \leq \sqrt{4 {( D - {D}_{1} )}^{2} + {( P - {P}_{1} )}^{2} + {( H - {H}_{1} )}^{2}} ,$$

where Ra 13.4 MPa^{0.5}, D₁=16.8 MPa^{0.5}, P₁=4.8 MPa^{0.5}, and H₁=13.0 MPa^{0.5}. The method may include combining the reaction product and the one or more solvents to form a premix consisting of the reaction product and the one or more solvents.

The one or more solvents may include, e.g., caprylic/capric triglyceride, isopropyl myristate, citronellol acetate, acetone, triglycerides, or a combination thereof. In some embodiments, at least some of the triglycerides provided by a vegetable oil. The reaction product may be present in an amount of 10-80% by weight of the cosmetic premix composition. The natural or food-derived oil may be linseed oil.

In various aspects, a cosmetic composition may be provided. The cosmetic composition may include a premix as well as one or more additional materials. The premix may include a reaction product of an acrylate and a natural or food-derived oil as disclosed herein. The premix may include one or more solvents of the reaction product, and one or more additional materials. Each solvent may have Hansen Solubility Parameters that satisfy

$$Ra \leq \sqrt{4 {( D - {D}_{1} )}^{2} + {( P - {P}_{1} )}^{2} + {( H - {H}_{1} )}^{2}} ,$$

where Ra=13.4 MPa^{0.5}, D₁=16.8 MPa^{0.5}, P₁=4.8 MPa^{0.5}, and H₁=13.0 MPa^{0.5}.

The one or more additional materials may include one or more additional solvents having Hansen Solubility Parameters that satisfy

$$Ra > \sqrt{4 {( D - {D}_{1} )}^{2} + {( P - {P}_{1} )}^{2} + {( H - {H}_{1} )}^{2}} ,$$

where Ra=13.4 MPa^{0.5}, D₁=16.8 MPa^{0.5}, P₁=4.8 MPa^{0.5}, and H₁=13.0 MPa^{0.5}. The one or more additional materials includes one or more pigments, a filler, a polyol, a polyester of polyglycerol having 2 to 10 glycerol units, a glyceryl ester of a fatty acid, or a combination thereof.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

As used herein, the term “free of (a component)” indicates the compositions do not contain the component in any measurable degree by standard means. As used herein, the term “substantially free of (a component)” indicates that the compositions contain no appreciable amount of the component, for example, no more than about 1% by weight, or no more than about 0.5% by weight, or no more than about 0.3% by weight, such as no more than about 0.1% by weight, based on the weight of the system or composition comprising the system and/or the composition according to embodiments of the disclosure.

In various aspects, a cosmetic premix composition may be provided.

### Reaction Product

The cosmetic premix composition may include a reaction product. The reaction product may be a reaction product of a natural or food-derived oil and a methacrylate or acrylate polymer. The reaction product may be a result of a reaction between poly (isobutyl methacrylate) and a natural or food derived oil. The natural or food derived oil may be a drying oil (such as linseed oil). Preferably, the reaction product is under the trade name MYCELX® from Mycelx Technologies Corporation of Gainesville, Georgia. See U.S. Pat. No. 5,698,139 and EP 0 871 593 BI for a description of MYCELX® materials.

The reaction product may be a reaction product of a natural or food-derived oil and a methacrylate or acrylate polymer. The reaction product may be a reaction product of a natural or food-derived oil and a methacrylate polymer. The natural or food-derived oil may be a drying oil or semi-drying oil, preferably wherein the natural or food-derived oil is linseed oil, sunflower oil, tung oil, fish oil, cottonseed oil, soybean oil, or a combination thereof. The natural or food-derived oil may be linseed oil. The polymer may be derived from monomers including, e.g., isobutyl methacrylate, methyl methacrylate, ethyl methacrylate, n-butyl methacrylate, or a combination thereof. The polymer may be an isobutyl methacrylate polymer. The reaction product may be a reaction product of linseed oil and poly (isobutyl methacrylate).

The reaction product may form, e.g., a hydrophobic polymer.

The reaction product may be present in a total amount of no more than 80% by weight of the cosmetic premix composition. The reaction product may be present in a total amount of no more than 75% by weight of the cosmetic premix composition. The reaction product may be present in a total amount of no more than 70% by weight of the cosmetic premix composition. The reaction product may be present in a total amount of no more than 65% by weight of the cosmetic premix composition. The reaction product may be present in a total amount of no more than 60% by weight of the cosmetic premix composition. The reaction product may be present in a total amount of no more than 55% by weight of the cosmetic premix composition. The reaction product may be present in a total amount of no more than 50% by weight of the cosmetic premix composition. The reaction product may be present in a total amount of no more than 45% by weight of the cosmetic premix composition. The reaction product may be present in a total amount of no more than 40% by weight of the cosmetic premix composition. The reaction product may be present in a total amount of no more than 35% by weight of the cosmetic premix composition. The reaction product may be present in a total amount of no more than 30% by weight of the cosmetic premix composition. The reaction product may be present in a total amount of no more than 25% by weight of the cosmetic premix composition. The reaction product may be present in a total amount of no more than 20% by weight of the cosmetic premix composition. The reaction product may be present in a total amount of no more than 15% by weight of the cosmetic premix composition. The reaction product may be present in a total amount of at least 10% by weight of the cosmetic premix composition. The reaction product may be present in a total amount of at least 15% by weight of the cosmetic premix composition. The reaction product may be present in a total amount of at least 20% by weight of the cosmetic premix composition. The reaction product may be present in a total amount of at least 25% by weight of the cosmetic premix composition. The reaction product may be present in a total amount of at least 30% by weight of the cosmetic premix composition. The reaction product may be present in a total amount of at least 35% by weight of the cosmetic premix composition. The reaction product may be present in a total amount of at least 40% by weight of the cosmetic premix composition. The reaction product may be present in a total amount of at least 45% by weight of the cosmetic premix composition. The reaction product may be present in a total amount of at least 50% by weight of the cosmetic premix composition. The reaction product may be present in a total amount of at least 55% by weight of the cosmetic premix composition. The reaction product may be present in a total amount of at least 60% by weight of the cosmetic premix composition. The reaction product may be present in a total amount of at least 65% by weight of the cosmetic premix composition. The reaction product may be present in a total amount of at least 70% by weight of the cosmetic premix composition. The reaction product may be present in a total amount of at least 75% by weight of the cosmetic premix composition.

### Solvent for the Reaction Product

The cosmetic premix composition may include a solvent for the reaction product. The solvent may be a single solvent. The solvent may be a plurality of solvents. Each solvent may have Hansen Solubility Parameters within a designated range. Solvents having a hydrogen bonding component (D), a polar component (P), and a dispersion component (H) may be selected, where the Hansen Solubility Parameters satisfy

$$Ra \leq \sqrt{4 {( D - {D}_{1} )}^{2} + {( P - {P}_{1} )}^{2} + {( H - {H}_{1} )}^{2}} \qquad ( Eq . 1 )$$

where Ra=13.4 MPa^{0.5}, D₁=16.8 MPa^{0.5}, P₁=4.8 MPa^{0.5}, and H₁=13.0 MPa^{0.5}. In some preferred embodiments, each solvent must have solubility parameters that satisfy that same equation, but where Ra=9.9 MPa^{0.5}, D₁=16.4 MPa^{0.5}, P₁=5.0 MPa^{0.5}, and H₁=11.7 MPa^{0.5}.

The solvent may be an oil. The term “oil” is intended to mean a non-aqueous compound, non-miscible in water, liquid at 25° C. and atmospheric pressure (760 mmHg; 1.013.105 Pa). The solvent may be a non-silicone oil (e.g., an oil that does not containing a silicon atom, and in particular contain a Si—O group).

Non-limiting examples of the solvent include caprylic/capric triglyceride, isopropyl myristate, and/or citronellol acetate or polycitronellol acetate. The solvent may include acetone. The solvent may include triglycerides. The solvent may include a triglycerides-containing oil (such as a vegetable oil). The solvent may include oleic acid and/or an oleic acid-containing oil.

Tables 1 and 2, below, shows values of D, P, and H, as well as Ra values for the allowable range as well as the preferred range, for several preferred solvents.

TABLE 1
(The D, P, and H were determined
based on compatibility at 55° C.)

| | | | | Ra | Ra |
|---|---|---|---|---|---|
| | | | | (Allowable | (Preferred |
| Material | D | P | H | Range) | Range) |
| Ethanol | 15.8 | 8.8 | 19.4 | 7.81 | 8.67 |
| Octyldodecanol | 16.1 | 2.2 | 7.4 | 6.33 | 5.17 |
| Isopropyl Myristate | 15.9 | 2.1 | 2.8 | 10.70 | 9.41 |
| Isopropyl Alcohol | 15.8 | 6.1 | 6.4 | 7.02 | 5.54 |
| Phenoxyethanol | 17.8 | 5.7 | 14.3 | 2.55 | 3.88 |
| CCTG | 18.22 | 5.39 | 14.74 | 3.38 | 4.76 |
| Castor Oil | 15.9 | 4.6 | 12 | 2.07 | 1.12 |
| Polycitronellol Acetate | 16.4 | 3 | 4.2 | 9.02 | 7.76 |

TABLE 2
(The D, P, and H were determined
based on compatibility at 25° C.)

| | | | | Ra | Ra |
|---|---|---|---|---|---|
| | | | | (Allowable | (Preferred |
| Material | D | P | H | Range) | Range) |
| Acetone | 15.5 | 10.4 | 7 | 8.61 | 7.38 |
| Oleic Acid | 16 | 2.8 | 6.2 | 7.27 | 5.98 |

In some embodiments, if triglycerides are utilized, at least some of the triglycerides may be provided by a vegetable oil (e.g., an triglycerides-containing oil). The vegetable oil may be a seed or nut oil. The vegetable oil may have an triglycerides content of at least 20% by weight of the vegetable oil. The vegetable oil may include sunflower oil, soybean oil, macadamia nut oil, and/or avocado oil. In some embodiments, the cosmetic premix composition may include macadamia nut oil, and may be free, or substantially free, of other vegetable oils.

The solvent(s) may be present in a total amount of at least 20% by weight of the cosmetic premix composition. The solvent(s) may be present in a total amount of at least 25% by weight of the cosmetic premix composition. The solvent(s) may be present in a total amount of at least 30% by weight of the cosmetic premix composition. The solvent(s) may be present in a total amount of at least 35% by weight of the cosmetic premix composition. The solvent(s) may be present in a total amount of at least 40% by weight of the cosmetic premix composition. The solvent(s) may be present in a total amount of at least 45% by weight of the cosmetic premix composition. The solvent(s) may be present in a total amount of at least 50% by weight of the cosmetic premix composition. The solvent(s) may be present in a total amount of at least 55% by weight of the cosmetic premix composition. The solvent(s) may be present in a total amount of at least 60% by weight of the cosmetic premix composition. The solvent(s) may be present in a total amount of at least 65% by weight of the cosmetic premix composition. The solvent(s) may be present in a total amount of at least 70% by weight of the cosmetic premix composition. The solvent(s) may be present in a total amount of at least 75% by weight of the cosmetic premix composition. The solvent(s) may be present in a total amount of at least 80% by weight of the cosmetic premix composition. The solvent(s) may be present in a total amount of at least 85% by weight of the cosmetic premix composition. The solvent(s) may be present in a total amount of no more than 90% by weight of the cosmetic premix composition. The solvent(s) may be present in a total amount of no more than 85% by weight of the cosmetic premix composition. The solvent(s) may be present in a total amount of no more than 80% by weight of the cosmetic premix composition. The solvent(s) may be present in a total amount of no more than 75% by weight of the cosmetic premix composition. The solvent(s) may be present in a total amount of no more than 70% by weight of the cosmetic premix composition. The solvent(s) may be present in a total amount of no more than 65% by weight of the cosmetic premix composition. The solvent(s) may be present in a total amount of no more than 60% by weight of the cosmetic premix composition. The solvent(s) may be present in a total amount of no more than 55% by weight of the cosmetic premix composition. The solvent(s) may be present in a total amount of no more than 50% by weight of the cosmetic premix composition. The solvent(s) may be present in a total amount of no more than 45% by weight of the cosmetic premix composition. The solvent(s) may be present in a total amount of no more than 40% by weight of the cosmetic premix composition. The solvent(s) may be present in a total amount of no more than 35% by weight of the cosmetic premix composition. The solvent(s) may be present in a total amount of no more than 30% by weight of the cosmetic premix composition. The solvent(s) may be present in a total amount of no more than 25% by weight of the cosmetic premix composition.

In general, the premix composition may comprise or consist of the reaction product and the solvent. Preferably, the premix composition consists of the reaction product and the solvent. In some embodiments, the premix composition may be substantially free of any other material. In some embodiments, the premix composition may include at least one other material that is miscible or soluble in the solvent.

### Method

In various aspects, a method of manufacturing a cosmetic composition may be provided. The method may include forming a premix. The premix may be formed by first providing a reaction product of an acrylate and a natural or food-derived oil, as disclosed herein. The method of forming the premix may include providing one or more solvents of the reaction product, as disclosed herein. As disclosed herein, each solvent may have Hansen Solubility Parameters that satisfy Eq. 1, where Ra=13.4 MPa^{0.5}, D₁=16.8 MPa^{0.5}, P₁=4.8 MPa⁹⁵, and H₁=13.0 MPa^{0.5}. The method may include combining the reaction product and the one or more solvents to form a premix. The premix may comprise the reaction product and the one or more solvents. The premix may preferably consist of the reaction product and the one or more solvents.

The premix may then be placed into a storage container or vessel. The premix may then be used to combine with other materials as desired. The result of the mixing may be a cosmetic composition. Thus, in various embodiments, a cosmetic composition may be provided.

For example, in some embodiments, the method may include combining one or more additional materials with the premix. The amount of the one or more additional materials added may be selected such that the total amount of the reaction product in the resulting cosmetic composition may be no more than 5% by weight of the cosmetic composition. The amount of the one or more additional materials added may be selected such that the total amount of the reaction product in the resulting cosmetic composition may be no more than 4% by weight of the cosmetic composition. The amount of the one or more additional materials added may be selected such that the total amount of the reaction product in the resulting cosmetic composition may be no more than 3% by weight of the cosmetic composition. The amount of the one or more additional materials added may be selected such that the total amount of the reaction product in the resulting cosmetic composition may be no more than 2% by weight of the cosmetic composition. The amount of the one or more additional materials added may be selected such that the total amount of the reaction product in the resulting cosmetic composition may be no more than 1% by weight of the cosmetic composition. The amount of the one or more additional materials added may be selected such that the total amount of the reaction product in the resulting cosmetic composition may be no more than 0.75% by weight of the cosmetic composition. The amount of the one or more additional materials added may be selected such that the total amount of the reaction product in the resulting cosmetic composition may be no more than 0.5% by weight of the cosmetic composition.

### Cosmetic Composition

In various aspects, a cosmetic composition may be provided. The cosmetic composition may exist any appropriate form, such as an emulsion (including a water-in-oil emulsion or an oil-in water emulsion), an anhydrous solution, etc., and may be a gel, lotion, crème, stick, etc.

The cosmetic composition may include an embodiment of a cosmetic premix composition as disclosed herein, and one or more additional materials. As such, the premix may include a reaction product of an acrylate and a natural or food-derived oil as disclosed herein. The premix may include one or more solvents of the reaction product. Each of the one or more solvents may, as disclosed herein, have Hansen Solubility Parameters that satisfy Eq. 1, where Ra=13.4 MPa^{0.5}, D₁=16.8 MPa^{0.5}, P₁=4.8 MPa^{0.5}, and H₁=13.0 MPa^{0.5}.

The reaction product may be present in a total amount of no more than 20% by weight of the cosmetic composition. The reaction product may be present in a total amount of no more than 19% by weight of the cosmetic composition. The reaction product may be present in a total amount of no more than 18% by weight of the cosmetic composition. The reaction product may be present in a total amount of no more than 17% by weight of the cosmetic composition. The reaction product may be present in a total amount of no more than 16% by weight of the cosmetic composition. The reaction product may be present in a total amount of no more than 15% by weight of the cosmetic composition. The reaction product may be present in a total amount of no more than 14% by weight of the cosmetic composition. The reaction product may be present in a total amount of no more than 13% by weight of the cosmetic composition. The reaction product may be present in a total amount of no more than 12% by weight of the cosmetic composition. The reaction product may be present in a total amount of no more than 11% by weight of the cosmetic composition. The reaction product may be present in a total amount of no more than 10% by weight of the cosmetic composition. The reaction product may be present in a total amount of no more than 9% by weight of the cosmetic composition. The reaction product may be present in a total amount of no more than 8% by weight of the cosmetic composition. The reaction product may be present in a total amount of no more than 7% by weight of the cosmetic composition. The reaction product may be present in a total amount of no more than 6% by weight of the cosmetic composition. The reaction product may be present in a total amount of no more than 5% by weight of the cosmetic composition. The reaction product may be present in a total amount of no more than 4% by weight of the cosmetic composition. The reaction product may be present in a total amount of no more than 3% by weight of the cosmetic composition. The reaction product may be present in a total amount of no more than 2.5% by weight of the cosmetic composition. The reaction product may be present in a total amount of no more than 2% by weight of the cosmetic composition. The reaction product may be present in a total amount of no more than 1.5% by weight of the cosmetic composition. The reaction product may be present in a total amount of no more than 1% by weight of the cosmetic composition. The reaction product may be present in a total amount of no more than 0.75% by weight of the cosmetic composition. The reaction product may be present in a total amount of no more than 0.5% by weight of the cosmetic composition. The reaction product may be present in a total amount of at least 0.1% by weight of the cosmetic composition. The reaction product may be present in a total amount of at least 0.5% by weight of the cosmetic composition. The reaction product may be present in a total amount of at least 1% by weight of the cosmetic composition.

### Additional Materials

The one or more additional materials may include one or more additional solvents having Hansen Solubility Parameters that around outside the range for solvents of the reaction product. That is, the one or more additional solvent(S) may have Hansen Solubility Parameters that satisfy

$$Ra > \sqrt{4 {( D - {D}_{1} )}^{2} + {( P - {P}_{1} )}^{2} + {( H - {H}_{1} )}^{2}} \qquad ( Eq . 2 )$$

where Ra=13.4 MPa^{0.5}, D₁=16.8 MPa^{0.5}, P₁=4.8 MPa^{0.5}, and H₁=13.0 MPa^{0.5}

Table 3, below, shows values of D, P, and H, as well as calculated Ra values for the allowable range as well as the preferred range, for several solvents that fall outside the allowable range.

TABLE 3
(The D, P, and H were determined
based on compatibility at 55° C.)

| | | | | Ra | Ra |
|---|---|---|---|---|---|
| | | | | (Allowable | (Preferred |
| Material | D | P | H | Range) | Range) |
| Propanediol | 16.8 | 13.5 | 23.2 | 13.41 | 14.32 |
| Glycerin | 17.4 | 11.3 | 27.2 | 15.66 | 16.85 |
| Water | 15.5 | 16 | 42.3 | 31.48 | 32.57 |
| Isohexadecane | 14.8 | 0.1 | 0.1 | 14.30 | 12.99 |
| Isododecane | 15.6 | 0.1 | 0.1 | 13.94 | 12.69 |
| Squalane | 15.9 | 0.1 | 0.1 | 13.85 | 12.63 |
| Undecane (and) | 15.94 | 0.03 | 0.03 | 13.93 | 12.72 |
| Tridecane | | | | | |

It should be noted that these additional solvents may not be soluble with the reaction product by itself. Non-limiting examples of such components include water, glycerin, and propanediol. Additional examples may include various alkanes, such as C10-C18 alkanes, which may be branched alkanes such as isohexadecane, isododecane, or squalane, or linear (non-branched) alkanes such as decane, undecane, or tridecane.

As seen in Table 4, when 10% of the reaction product was mixed with 90% of the additional solvent material at 55° C., various instabilities were detected during stability testing.

TABLE 4
(Visible Stability Results)

| | Visible |
|---|---|
| Material | Stability Result |
| Propanediol | Separation |
| Glycerin | Separation |
| Water | Separation |
| Isohexadecane | Precipitation |
| Isododecane | Precipitation |
| Squalane | Precipitation |
| Undecane (and) | Precipitation |
| Tridecane | |

The additional solvent(s) may be present in a total amount of at least 1% by weight of the cosmetic composition. The additional solvent(s) may be present in a total amount of at least 3% by weight of the cosmetic composition. The additional solvent(s) may be present in a total amount of at least 5% by weight of the cosmetic composition. The additional solvent(s) may be present in a total amount of at least 10% by weight of the cosmetic composition. The additional solvent(s) may be present in a total amount of at least 15% by weight of the cosmetic composition. The additional solvent(s) may be present in a total amount of at least 20% by weight of the cosmetic composition. The additional solvent(s) may be present in a total amount of at least 25% by weight of the cosmetic composition. The additional solvent(s) may be present in a total amount of at least 30% by weight of the cosmetic composition. The additional solvent(s) may be present in a total amount of no more than 99% by weight of the cosmetic composition. The additional solvent(s) may be present in a total amount of no more than 90% by weight of the cosmetic composition. The additional solvent(s) may be present in a total amount of no more than 80% by weight of the cosmetic composition. The additional solvent(s) may be present in a total amount of no more than 70% by weight of the cosmetic composition. The additional solvent(s) may be present in a total amount of no more than 60% by weight of the cosmetic composition. The additional solvent(s) may be present in a total amount of no more than 50% by weight of the cosmetic composition. The additional solvent(s) may be present in a total amount of no more than 40% by weight of the cosmetic composition.

### Polyol

The additional solvent(s) may include at least one polyol. The polyol(s) may be present in a total amount of at least 1% by weight of the cosmetic composition. The polyol(s) may be present in a total amount of at least 2% by weight of the cosmetic composition. The polyol(s) may be present in a total amount of at least 3% by weight of the cosmetic composition. The polyol(s) may be present in a total amount of at least 5% by weight of the cosmetic composition. The polyol(s) may be present in a total amount of no more than 30% by weight of the cosmetic composition. The polyol(s) may be present in a total amount of no more than 25% by weight of the cosmetic composition. The polyol(s) may be present in a total amount of no more than 20% by weight of the cosmetic composition. The polyol(s) may be present in a total amount of no more than 15% by weight of the cosmetic composition. The polyol(s) may be present in a total amount of no more than 10% by weight of the cosmetic composition. The polyol(s) may be present in a total amount of no more than 5% by weight of the cosmetic composition. The cosmetic composition may be free, or substantially free, of a polyol. The cosmetic premix composition may be free, or substantially free, of a polyol.

The polyol may include a non-sugar alcohol. The non-sugar alcohol may include at least one polyol between 2 to 20 carbon atoms, such as between 2 to 10 carbon atoms, such as between 2 to 6 carbon atoms. Non-limiting examples of a polyol include glycerol and glycols, such as propylene glycol, butylene glycol, pentylene glycol, hexylene glycol, dipropylene glycol, diethylene glycol, glycol ethers, preferably mono-, di- or tripropylene glycol of alkyl(C₁-C₄) ether or mono-, di- or triethylene glycol of alkyl(C₁-C₄) ether, or mixtures thereof.

The non-sugar alcohol(s) may be present in a total amount of at least 1% by weight of the cosmetic composition. The non-sugar alcohol(s) may be present in a total amount of at least 2% by weight of the cosmetic composition. The non-sugar alcohol(s) may be present in a total amount of at least 3% by weight of the cosmetic composition. The non-sugar alcohol(s) may be present in a total amount of no more than 15% by weight of the cosmetic composition. The non-sugar alcohol(s) may be present in a total amount of no more than 10% by weight of the cosmetic composition. The non-sugar alcohol(s) may be present in a total amount of no more than 5% by weight of the cosmetic composition. The cosmetic composition may be free, or substantially free, of a non-sugar alcohol. The cosmetic premix composition may be free, or substantially free, of a non-sugar alcohol.

The polyol may include a sugar alcohol. The sugar alcohol may include a monosaccharide-polyol. The monosaccharide-polyol may be, e.g., mannitol, erythritol, xylitol, sorbitol, or a mixture thereof.

The sugar alcohol(s) may be present in a total amount of at least 1% by weight of the cosmetic composition. The sugar alcohol(s) may be present in a total amount of at least 2% by weight of the cosmetic composition. The sugar alcohol(s) may be present in a total amount of at least 3% by weight of the cosmetic composition. The sugar alcohol(s) may be present in a total amount of at least 4% by weight of the cosmetic composition. The sugar alcohol(s) may be present in a total amount of at least 5% by weight of the cosmetic composition. The sugar alcohol(s) may be present in a total amount of no more than 15% by weight of the cosmetic composition. The sugar alcohol(s) may be present in a total amount of no more than 10% by weight of the cosmetic composition. The sugar alcohol(s) may be present in a total amount of no more than 9% by weight of the cosmetic composition. The sugar alcohol(s) may be present in a total amount of no more than 8% by weight of the cosmetic composition. The sugar alcohol(s) may be present in a total amount of no more than 7% by weight of the cosmetic composition. The sugar alcohol(s) may be present in a total amount of no more than 6% by weight of the cosmetic composition. The sugar alcohol(s) may be present in a total amount of no more than 5% by weight of the cosmetic composition. The cosmetic composition may be free, or substantially free, of a sugar alcohol. The cosmetic premix composition may be free, or substantially free, of a sugar alcohol.

### Silicone Oil

The additional solvent(s) may include a silicone oil. As used herein, the term “silicone oil” refers to an oil containing at least one silicon atom, and in particular containing Si—O groups. The silicone oil may be present in a total amount of at least 0.1% by weight of the cosmetic composition. The silicone oil may be present in a total amount of at least 0.5% by weight of the cosmetic composition. The silicone oil may be present in a total amount of at least 1% by weight of the cosmetic composition. The silicone oil may be present in a total amount of no more than 5% by weight of the cosmetic composition. The silicone oil may be present in a total amount of no more than 2% by weight of the cosmetic composition. The silicone oil may be present in a total amount of no more than 1% by weight of the cosmetic composition. The cosmetic composition may be free, or substantially free, of a silicone oil. The cosmetic premix composition may be free, or substantially free, of a silicone oil.

The silicone oil may be present in a total amount (by weight % of the composition) less than the amount of the solvent of the reaction product. The silicone oil may be present in a total amount (by weight % of the composition) less than the amount of the reaction product.

The silicone oil(s) may include a non-volatile silicone oil. Non-limiting examples of non-volatile silicone oils include non-volatile polydimethylsiloxanes (PDMS); phenyl silicones such as phenyl trimethicones, phenyl dimethicones, diphenyl dimethicones, tri methyl penta phenyltrisiloxanes, tetra methyltetra phenyltrisiloxanes, trimethylsiloxyphenyl dimethicones, diphenylsiloxyphenyl trimethicones, or combinations thereof. The cometic composition may be free, or substantially free, of non-volatile silicone oils. The cometic premix composition may be free, or substantially free, of non-volatile silicone oils.

The silicone oil(s) may include volatile silicone oil. The volatile silicone oil may include a linear volatile silicone oil and/or a cyclic volatile silicone oil. Non-limiting examples of linear volatile silicone oils include octamethyltrisiloxane, decamethyltetrasiloxane, dodecamethylpentasiloxane, or combinations thereof. Non-limiting examples of cyclic volatile silicone oils include octamethylcyclotetrasiloxane, decamethylcyclopentasiloxane, dodecamethylcyclohexasiloxane, or combinations thereof.

The cosmetic composition may be free, or substantially free, of volatile silicone oils. The cosmetic premix composition may be free, or substantially free, of volatile silicone oils.

### Colorants

The one or more additional materials may include at least one cosmetically acceptable colorant, such as a pigment or dyestuff.

The term “pigment” means any pigment that gives color to keratin materials. Their solubility in water at 25° C. and atmospheric pressure (760 mmHg) is less than 0.05% by weight, preferably less than 0.01%.

Examples of suitable pigments include, but are not limited to, inorganic pigments, organic pigments, lakes, pearlescent pigments, iridescent or optically variable pigments, and mixtures thereof. The pigments may be chosen from the organic and/or mineral pigments known in the art. The pigments may be in the form of powder or of pigmentary paste. They may be coated or uncoated. Said pigments may optionally be surface-treated within the scope of the present invention but are not limited to treatments such as silicones, perfluorinated compounds, lecithin, and amino acids.

The pigments may be chosen, for example, from inorganic pigments, organic pigments, lakes, pigments with special effects such as nacres or glitter flakes, and mixtures thereof. Non-limiting examples of inorganic pigments include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The pigment may be an inorganic pigment. The term “inorganic pigment” refers to any pigment that satisfies the definition in Ullmann's encyclopedia in the chapter on inorganic pigments. Non-limiting examples include iron oxides, chromium oxides, manganese violet, ultramarine blue, chromium hydrate, ferric blue and titanium oxide.

The pigment may be an organic pigment. The term “organic pigment” refers to any pigment that satisfies the definition in Ullmann's encyclopedia in the chapter on organic pigments. Non-limiting examples include nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane and quinophthalone compounds.

In particular, the white or colored organic pigments may be chosen from carmine, carbon black, aniline black, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and pigments obtained by oxidative polymerization of indole or phenolic derivatives. Non-limiting examples of organic pigments and lakes include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12.085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75.570) and mixtures thereof.

The pigments in accordance with the invention may also be in the form of composite pigments. These composite pigments may be compounds especially of particles comprising a inorganic core, at least one binder for ensuring the binding of the organic pigments to the core, and at least one organic pigment at least partially covering the core.

The organic pigment may also be a lake. The term “lake” refers dyes adsorbed onto insoluble particles, the assembly thus obtained remaining insoluble during use.

The inorganic substrates onto which the dyes are adsorbed are, for example, alumina, silica, calcium sodium borosilicate or calcium aluminum borosilicate, and aluminum.

Among the dyes, mention may be made of cochineal carmine. Non-limiting examples of dyes include: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), D&C Blue 1 (CI 42 090). An example of a lake is D&C Red 7 (CI 15 850:1).

The pigment may also be a pigment with special effects. The term “pigments with special effects” refers pigments that generally create a non-uniform colored appearance (characterized by a certain shade, a certain vivacity and a certain lightness) that changes as a function of the conditions of observation (light, temperature, observation angles, etc.). They thus contrast with colored pigments that afford a standard uniform opaque, semi-transparent or transparent shade.

Several types of pigment with special effects exist: those with a low refractive index, such as fluorescent, photochromic or thermochromic pigments, and those with a higher refractive index, such as nacres or glitter flakes.

Examples of pigments with special effects that may be mentioned include nacreous pigments such as titanium mica coated with an iron oxide, mica coated with an iron oxide, mica coated with bismuth oxychloride, titanium mica coated with chromium oxide, titanium mica coated with an organic dye especially of the abovementioned type, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery color or tint.

Non-limiting examples of nacres include gold-colored nacres sold especially by the company Engelhard under the name Gold 222C (a CLOISONNE® pigment), Sparkle gold (a TIMICA® pigment), Gold 4504 (a CHROMA-LITER pigment) and Monarch gold 233X (a CLOISONNÉ® pigment); the bronze nacres sold especially by the company Merck under the name Bronze fine (17384) (a COLORONA® pigment) and Bronze (17353) (a COLORONA® pigment), by the company Eckart under the name PRESTIGE™ Bronze pigment and by the company Engelhard under the name Super bronze (a CLOISONNÉ® pigment); the orange nacres sold especially by the company Engelhard under the name Orange 363C (a CLOISONNE® pigment) and Orange MCR 101 and by the company Merck under the name Passion orange (a COLORONA® pigment) and Matte orange (17449); the brown nacres sold especially by the company Engelhard under the name Nu-antique copper 340XB (a CLOISONNE® pigment) and Brown CL4509 (a CHROMA-LITER pigment); the nacres with a copper tint sold especially by the company Engelhard under the name Copper 340A (a TIMICA® pigment) and by the company Eckart under the name Prestige Copper; the nacres with a red tint sold especially by the company Merck under the name Sienna fine (17386) (a COLORONA® pigment); the nacres with a yellow tint sold especially by the company Engelhard under the name Yellow (4502) (a CHROMA-LITE® pigment); the red nacres with a gold tint sold especially by the company Engelhard under the name Sunstone G012 (a GEMTONE® pigment); the black nacres with a gold tint sold especially by the company Engelhard under the name Nu antique bronze 240 AB (a TIMICA® pigment), the blue nacres sold especially by the company Merck under the name Matte blue (17433), Dark Blue (117324) (a COLORONA® pigment), the white nacres with a silvery tint sold especially by the company Merck under the name XIRONA® Silver pigment, and the golden-green pink-orange nacres sold especially by the company Merck under the name Indian summer (a XIRONA® pigment), and mixtures thereof.

In addition to nacres on a mica support, multilayer pigments based on synthetic substrates such as alumina, silica, sodium calcium borosilicate or calcium aluminum borosilicate, and aluminum, may be envisaged.

Non-limiting examples of pearlescent pigments include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

Mention may also be made of pigments with an interference effect that are not fixed onto a substrate, for instance liquid crystals or holographic interference glitter flakes. Pigments with special effects also comprise fluorescent pigments, whether these are substances that are fluorescent in daylight or that produce an ultraviolet fluorescence, phosphorescent pigments, photochromic pigments, thermochromic pigments and quantum dots.

The variety of pigments that may be used in the present invention makes it possible to obtain a wide range of colors, and also particular optical effects such as metallic effects or interference effects.

The effective diameter of the pigment particles may generally be between 10 nm and 200 μm, such as between 20 nm and 80 μm, such as between 30 nm and 50 μm.

The pigment may be present in a total amount of no more than 20% by weight of the cosmetic composition. The pigment may be present in a total amount of no more than 15% by weight of the cosmetic composition. The pigment may be present in a total amount of no more than 10% by weight of the cosmetic composition. The pigment may be present in a total amount of no more than 8% by weight of the cosmetic composition. The pigment may be present in a total amount of no more than 6% by weight of the cosmetic composition. The pigment may be present in a total amount of no more than 5% by weight of the cosmetic composition. The pigment may be present in a total amount of at least 0.1% by weight of the cosmetic composition. The pigment may be present in a total amount of at least 1% by weight of the cosmetic composition. The pigment may be present in a total amount of at least 2% by weight of the cosmetic composition. The pigment may be present in a total amount of at least 3% by weight of the cosmetic composition. The pigment may be present in a total amount of at least 4% by weight of the cosmetic composition. The pigment may be present in a total amount of at least 5% by weight of the cosmetic composition.

The colorant may comprise or consist of a pigment. The pigment may be a single pigment. The pigment may be a plurality of pigments.

In some embodiments, titanium dioxide may be a primary component of the pigment(s). For example, in some embodiments, the pigments may be comprised by titanium dioxide, where the titanium dioxide may be present in an amount of at least 55% by weight of the pigment. The titanium dioxide is present in an amount of at least 75% by weight of the pigment. The titanium dioxide is present in an amount of at least 80% by weight of the pigment. The titanium dioxide is present in an amount of at least 85% by weight of the pigment. The titanium dioxide is present in an amount of at least 90% by weight of the pigment.

### Filler

The one or more additional materials may include one or more fillers. Non-limiting examples of fillers include silica powder; talc; polyamide particles and especially those sold under the name ORGASOL® powders by the company Atochem; polyethylene powders; microspheres based on acrylic copolymers, such as those based on ethylene glycol dimethacrylate/lauryl methacrylate copolymer sold by the company Dow Corning under the name POLYTRAP® systems; expanded powders such as hollow microspheres and especially the microspheres sold under the name EXPANCEL® thermoplastic microspheres by the company Kemanord Plast or under the name MICROSPHERE® F 80 ED microcapsules by the company Matsumoto; powders of natural organic materials such as crosslinked or noncrosslinked corn starch, wheat starch or rice starch, such as the powders of starch crosslinked with octenyl succinate anhydride, sold under the name DRY-FLO® starch by the company National Starch; silicone resin microbeads such as those sold under the name TOSPEARL® microspheres by the company Toshiba Silicone: clays (bentone, laponite, saponite, etc.); and mixtures thereof.

The filler may include a clay (such as kaolin, disteardimonium hectorite, or a combination thereof), which may include a swellable clay. By “swellable clay” it is meant a clay material that is capable of swelling in water. An example of a swellable clay are smectite clays. The crystal structure of the smectite group, is an octahedral alumina sheet between two tetrahedral silica sheets. In one notable embodiment, the swellable clay is bentonite. Bentonite is a rock formed of highly colloidal and plastic clays composed mainly of montmorillonite, a clay mineral of the smectite group, and is produced by in situ devitrification of volcanic ash. In addition to montmorillonite, bentonite may contain feldspar, cristobalite, and crystalline quartz. Bentonite has an ability to form thixotrophic gels with water, an ability to absorb large quantities of water. Variations in interstitial water and exchangeable cations in the interlayer space affect the properties of bentonite and thus the commercial uses of the different types of bentonite.

One notable swellable clay suitable for use in the cosmetic composition is BENTONE GEL® ISD V dispersion, commercially available from Elementis Specialties, East Windsor, New Jersey. BENTONE GEL® GTCC V dispersion is a dispersion of organically (disterammonium) modified hectorite in isododecane with added propylene carbonate.

The fillers may be present in the cosmetic compositions of the present invention in a total amount of at least 1% by weight. The fillers may be present in the cosmetic compositions of the present invention in a total amount of at least 1.5% by weight. The fillers may be present in the cosmetic compositions of the present invention in a total amount of at least 2% by weight. The fillers may be present in the cosmetic compositions of the present invention in a total amount of at least 2.5% by weight. The fillers may be present in the cosmetic compositions of the present invention in a total amount of at least 3% by weight. The fillers may be present in the cosmetic compositions of the present invention in a total amount of no more than 10% by weight. The fillers may be present in the cosmetic compositions of the present invention in a total amount of no more than 10% by weight. The fillers may be present in the cosmetic compositions of the present invention in a total amount of at least 1% by weight.

A carbonate, such as propylene carbonate or calcium carbonate, may be present. If present, the carbonate may be present in an amount such that the ratio by weight of swellable clay to propylene carbonate is about 2:1 to about 5:1.

While in certain embodiments, certain fillers such as hydrophobic silica aerogels may be included. In certain other embodiments, the cosmetic compositions may be substantially free of hydrophobic silica aerogels. The cosmetic premix compositions may free or substantially free of hydrophobic silica aerogels.

Silica aerogels are porous materials obtained by replacing (by drying) the liquid component of a silica gel with air. They are generally synthesized via a sol-gel process in liquid medium and then dried, usually by extraction of a supercritical fluid, the one most commonly used being supercritical CO₂. This type of drying makes it possible to avoid shrinkage of the pores and of the material. The sol-gel process and the various drying processes are described in detail in Brinker C J., and Scherer G. W., Sol-Gel Science: New York: Academic Press, 1990. Silica aerogels, in general, have been disclosed in U.S. Pat. No. 9,320,689, the entire content of which is hereby incorporated by reference.

As hydrophobic silica aerogels that may be used in the invention, examples that may be mentioned include the aerogel sold under the name VM-2260 (INCI name: Silica silylate), by the company Dow Corning, the particles of which have a mean size of about 1000 microns and a specific surface area per unit of mass ranging from 600 to 800 m²/g.

Mention may also be made of the aerogels sold by the company Cabot under the references Aerogel TLD 201, Aerogel OGD 201, Aerogel TLD 203, ENOVA® Aerogel MT 1100 silica aerogel, ENOVA® Aerogel MT 1200 silica aerogel.

Use will be made more particularly of the aerogel sold under the name VM-2270 (INCI name: Silica silylate), by the company Dow Corning, the particles of which have a mean size ranging from 5-15 microns and a specific surface area per unit of mass ranging from 600 to 800 m²/g.

The silica aerogel particles if used can be used in the cosmetic compositions from 0.1% to about 8% by weight, preferably from about 0.1%, 0.2%, or 0.5% to about 0.5%, 1% 2% or 5% by weight, all weights based on the weight of the cosmetic composition.

### Emulsifiers

The one or more additional materials may include one or more emulsifiers. As used herein, the term “emulsifier” refers to any substance or agent that aids in the formation of an emulsion. As used herein, the term “emulsion” refers to a generally stable and homogeneous mixture of two liquids that do not normally mix (i.e., they are immiscible between themselves), such as vegetable oil and water. Emulsions can be true colloids or less stable mixtures, which tend to separate in a short time. An emulsion can often be broken down (i.e. the liquids separated) by factors such as mechanical manipulation, chemical effects and/or time.

The emulsifier may be a surfactant. As used herein, the term “surfactant” refers to an amphiphilic molecule, i.e. a molecule that has two parts of different polarity, one generally being lipophilic (soluble or dispersible in an oily phase), and the other being hydrophilic (soluble or dispersible in water), and which is preferably capable of reducing the surface tension of water. Surfactants are characterized by their HLB (hydrophilic-lipophilic balance) value, the HLB being the ratio between the hydrophilic part and the lipophilic part in the molecule. The term “HLB” is well known to those skilled in the art and is described, for example, in “The HLB system. A time-saving guide to Emulsifier Selection” (published by ICI Americas Inc., 1984). For the emulsifying surfactants, the HLB generally ranges from 3 to 8 for the preparation of W/O emulsions and from 8 to 18 for the preparation of O/W emulsions. The HLB of the surfactant(s) used according to the invention may be determined via the Griffin method or the Davies method.

The emulsifier may comprise or consist of a polyester of polyglycerol having 2 to 10 glycerol units (such as polyglyceryl-6 polyricinoleate).

The emulsifier(s) may be present in a total amount of no more than 15% by weight of the cosmetic composition. The emulsifier(s) may be present in a total amount of no more than 10% by weight of the cosmetic composition. The emulsifier(s) may be present in a total amount of no more than 8% by weight of the cosmetic composition. The emulsifier(s) may be present in a total amount of no more than 7% by weight of the cosmetic composition. The emulsifier(s) may be present in a total amount of no more than 6% by weight of the cosmetic composition. The emulsifier(s) may be present in a total amount of at least 1% by weight of the cosmetic composition. The emulsifier(s) may be present in a total amount of at least 2% by weight of the cosmetic composition. The emulsifier(s) may be present in a total amount of at least 3% by weight of the cosmetic composition. The emulsifier(s) may be present in a total amount of at least 4% by weight of the cosmetic composition. The emulsifier(s) may be present in a total amount of at least 5% by weight of the cosmetic composition.

The cosmetic composition may be free, or substantially free, of an emulsifier. The cosmetic composition may be free, or substantially free, of a surfactant. The cosmetic premix composition may be free, or substantially free, of an emulsifier. The cosmetic premix composition may be free, or substantially free, of a surfactant.

### Thickening Agent

The one or more additional materials may include at least one thickening agent. Non-limiting examples include those conventionally used in cosmetics, such as polymers of natural origin and synthetic polymers. For example, nonionic, anionic, cationic, amphiphilic, and amphoteric polymers, and other known rheology modifiers, such as cellulose or a cellulose-based thickener, may be used.

According to certain embodiments, the thickening agent may include a cellulose-based thickener. Suitable cellulose-based compounds include, but are not limited to, cellulose polymers, such as, for example, hydroxyethylcellulose, hydroxypropylcellulose, methylcellulose, and ethylhydroxyethylcellulose. Certain notable cellulose derivatives include hydroxyl-modified cellulose polymers such as Hydroxyethylcellulose, e.g., those having a molecular weight over 500,000 daltons such as NATROSOL® 250 HHR hydroxethylcellulose and Hydroxypropyl cellulose, e.g., KLUCEL® MF hydroxypropyl cellulose-both available from Ashland of Covington, Ky.

According to other embodiments, the thickening agent may include a polysaccharide.

In general, polysaccharides may be divided into several categories. Polysaccharides that are suitable for use in the invention may be homopolysaccharides such as fructans, glucans, galactans and mannans or heteropolysaccharides such as hemicellulose.

The polysaccharides may include linear polysaccharides such as pullulan or branched polysaccharides such as gum arabic and amylopectin, or mixed polysaccharides such as starch. Suitable polysaccharides may be starchy polysaccharides. Starchy polysaccharides include, but are not limited to, native starches, modified starches and particulate starches.

According to other embodiments, the thickening agent may include an acrylic thickening agent (acrylic thickener) or an acrylamide thickening agent (acrylamide thickener).

“Acrylic thickening agent” or “acrylic thickener” as used herein refers to polymers based upon one or more (meth)acrylic acid (and corresponding (meth)acrylate) monomers or similar monomers.

“Acrylamide thickening agent” or “acrylamide thickener” as used herein refers to polymers based upon one or more acrylamide monomers or similar monomers.

According to other embodiments, the thickening agent may include at least one monomer performing a weak acid function such as, for example, acrylic acid, methacrylic acid, itaconic acid, crotonic acid, maleic acid and/or fumaric acid.

According to other embodiments, the thickening agent may include at least one monomer performing a strong acid function such as, for example, monomers having a function of the sulfonic acid type or phosphonic acid type, such as 2-acrylamido-2-methylpropane sulfonic acid (AMPS).

According to other embodiments, the thickening agent may be crosslinked (or branched). Non-limiting examples of acceptable crosslinking agents include methylene bisacrylamide (MBA), ethylene glycol diacrylate, polyethylene glycol dimethacrylate, diacrylamide, cyanomethacrylate, vinyloxyethacrylate or methacrylate, formaldehyde, glyoxal, and compositions of the glycidylether type such as ethyleneglycol diglycidylether, or epoxides.

Specific non-limiting examples of suitable thickening agents include homopolymers or copolymers of acrylic or methacrylic acids or the salts thereof and the esters thereof, polyacrylates and polymethacrylates, polyacrylic acids, polyacrylamides, copolymers of acrylic acid and of acrylamide sold in the form of the sodium salt thereof, sodium polymethacrylate, and the sodium salts of polyhydroxycarboxylic acids, optionally crosslinked and/or neutralized 2-acrylamido-2-methylpropanesulphonic acid polymers and copolymers, for instance poly (2-acrylamido-2-methylpropanesulphonic acid), crosslinked anionic copolymers of acrylamide and of AMPS, e.g. in the form of a water-in-oil emulsion, such as those sold under the name SEPIGEL® 305 (CTFA name: Polyacrylamide/C13-14 Isoparaffin/Laureth-7) and under the name SIMULGEL® 600 (INCI name: Acrylamide/Sodium acryloyldimethyltaurate copolymer/Isohexa-decane/Polysorbate 80) by SEPPIC, polyacrylic acid/alkyl acrylate copolymers of PEMULEN® copolymer type, sodium acrylate/sodium acryloyldimethyl taurate such as that sold under the INCI name Sodium Acrylate/Sodium Acryloyldimethyl Taurate Copolymer & Hydrogenated Polydecene & Sorbitan Laurate & Trideceth-6 which is marketed by Lonza, Allendale, N.J., USA under the tradename ViscUp® EZ. Certain especially notable acrylic thickeners are selected from the group of: Acrylamide/Sodium Acryloyldimethyl Taurate Copolymer such as those provided in Isohexadecane & Polysorbate 80 as SIMULGEL™ 600 and SIMULGEL™ 800 (Seppic, Inc.); Polyacrylamide provided with C13-14 isoparaffin and laureth-7 available as SEPIGEL 305TM (Seppic, Inc.); and Polyacrylate Crosspolymer-6 available as SEPIMAX ZEN (Seppic, Inc.). In certain embodiments, the thickening agent is selected from polyacrylamides and water-soluble cellulose polymers (such as hydroxypropylmethylcellulose, ethylcellulose, and/or hydroxypropylcellulose), and combinations thereof.

While clays and silicas may be considered thickening agents, for the purposes of this disclosure, the thickening agent may be free of clays and/or silicas, and the cosmetic composition may only contain clays or silicas as fillers.

The thickening agent(s) may be present in a total amount at least 0.05% by weight of the cosmetic composition. The thickening agent(s) may be present in a total amount at least 0.1% by weight of the cosmetic composition. The thickening agent(s) may be present in a total amount at least 0.5% by weight of the cosmetic composition. The thickening agent(s) may be present in a total amount at least 1% by weight of the cosmetic composition. The thickening agent(s) may be present in a total amount at least 1.5% by weight of the cosmetic composition. The thickening agent(s) may be present in a total amount at least 2% by weight of the cosmetic composition. The thickening agent(s) may be present in a total amount at least 2.5% by weight of the cosmetic composition. The thickening agent(s) may be present in a total amount at least 3% by weight of the cosmetic composition. The thickening agent(s) may be present in a total amount no more than 15% by weight of the cosmetic composition. The thickening agent(s) may be present in a total amount no more than 10% by weight of the cosmetic composition. The thickening agent(s) may be present in a total amount no more than 8% by weight of the cosmetic composition. The thickening agent(s) may be present in a total amount no more than 6% by weight of the cosmetic composition. The thickening agent(s) may be present in a total amount no more than 5% by weight of the cosmetic composition. The thickening agent(s) may be present in a total amount no more than 4% by weight of the cosmetic composition. The thickening agent(s) may be present in a total amount no more than 3% by weight of the cosmetic composition.

### Film Former

The one or more additional materials may include a film former. As used herein, the term “film former” refers to a polymer or resin that leaves a film on the substrate to which it is applied, for example, after a solvent accompanying the film former has evaporated, absorbed into and/or dissipated on the substrate.

Non-limiting examples of a film former include acrylic acid/isobutyl acrylates/isobornyl acrylate copolymer, trimethylsiloxysilicate, acrylates/isobornyl acrylate copolymer, norbornene/tris (trimethylsiloxy) silylnorbornene copolymer, acrylate/polytrimethyl siloxymethacrylate copolymer, C30-45 alkyldimethylsilylpolypropylsilsequioxane, trimethylsilsesquioxane, polypropylsilsesquioxane, acrylates/dimethicone copolymer, octylacrylamide/acrylates/butylaminoethyl methacrylate copolymer, VA/butyl maleate/isobornyl acrylate copolymer, acrylates/t-butylacrylamide copolymer, polyvinylpyrrolidone/vinyl acetate copolymer, triacontanyl PVP copolymer, acrylates/dimethylaminoethyl methacrylate copolymer, or a combination thereof.

The film former(s) may be present in a total amount of at least 0.5% by weight of the cosmetic composition. The film former(s) may be present in a total amount of at least 1% by weight of the cosmetic composition. The film former(s) may be present in a total amount of no more than 5% by weight of the cosmetic composition. The film former(s) may be present in a total amount of no more than 3% by weight of the cosmetic composition. The cosmetic composition may be free or substantially free of a film former. The cosmetic premix composition may be free or substantially free of a film former.

### Other Materials

The one or more additional materials may include one or more other materials, such as materials known to be used in cosmetics, such as a preservative, antioxidant, a chelating agent, a fragrance, a coating material, or a combination thereof.

The cosmetic composition may include other materials in a total amount of no more than 5% by weight of the cosmetic composition. The cosmetic composition may include no more than 4% by weight of other materials. The cosmetic composition may include no more than 3% by weight of other materials. The cosmetic composition may include no more than 2% by weight of other materials. The cosmetic composition may be free or substantially free of other materials.

### Example 1

Various premix compositions may be created by mixing the reaction product with one or more solvents as disclosed herein. Examples of such premixes can be seen in Table 5 below.

TABLE 5
Exemplary Premix Compositions (EPC),
all listed values are % w/w.

| Material | EPC1 | EPC2 | EPC3 | EPC4 | EPC5 |
|---|---|---|---|---|---|
| MYCELX ® material¹ | 10% | 10% | 10% | 10% | 10% |
| Isopropyl Myristate | 90% | | 45% | 40% | 35% |
| CCTG | | 90% | 45% | 40% | 35% |
| Polycitronellol Acetate | | | | 10% | 5% |
| Ethanol | | | | | 5% |

¹A reaction product from Mycelx Technologies Corporation, resulting of reaction from poly(isobutyl methacrylate) and the linseed oil.

### Example 2

A two-step makeup remover can be provided, as shown in Table 6, below. The reaction product and solvent (here, polycitronellol acetate) were formed into a premix as disclosed herein, and then isododecane was mixed into the premix until homogenous, forming a clear (transparent) composition.

TABLE 6
Composition Two-Step Makeup Remover.

| Material | E1 |
|---|---|
| First Step Composition | |
| MYCELX ® material¹ | 28.6% |
| Polycitronellol Acetate | 57.1% |
| Isododecane | 14.3% |
| Second Step Composition | |
| Mineral Oil | 100% |

¹A reaction product from Mycelx Technologies Corporation, resulting of reaction from poly(isobutyl methacrylate) and the linseed oil.

It will be understood that the second step composition may be, e.g., one or more alkanes such as mineral oil, squalene, etc. The first step composition is applied to skin, and after the second step composition is applied over the first step composition, the reaction product will pill, allowing for easy removal.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed is:

1. A cosmetic premix composition, consisting of:
a reaction product of a natural or food-derived oil and a methacrylate or acrylate polymer; and
one or more solvents, each solvent having Hansen Solubility Parameters that satisfy
$$Ra \leq \sqrt{4 {( D - {D}_{1} )}^{2} + {( P - {P}_{1} )}^{2} + {( H - {H}_{1} )}^{2}} ,$$
where Ra=13.4 MPa^{0.5}, D₁=16.8 MPa^{0.5}, P₁=4.8 MPa^{0.5}, and H₁=13.0 MPa^{0.5}.

2. The cosmetic premix composition of claim 1, wherein the one or more solvents comprises caprylic/capric triglyceride, isopropyl myristate, citronellyl acetate, acetone, triglycerides, a triglycerides-containing oil, or a combination thereof.

3. The cosmetic premix composition of claim 2, wherein at least some triglycerides are provided by a vegetable oil.

4. The cosmetic premix composition of claim 1, wherein the reaction product is present in an amount of 10-80% by weight of the cosmetic premix composition.

5. The cosmetic makeup composition of claim 1, wherein the natural or food-derived oil is linseed oil.

6. A method of manufacturing a cosmetic composition, comprising:
providing a reaction product of a natural or food-derived oil and a methacrylate or acrylate polymer;
providing one or more solvents, each solvent having Hansen Solubility Parameters that satisfy
$$Ra \leq \sqrt{4 {( D - {D}_{1} )}^{2} + {( P - {P}_{1} )}^{2} + {( H - {H}_{1} )}^{2}} ,$$
where Ra=13.4 MPa^{0.5}, D₁=16.8 MPa^{0.5}, P₁=4.8 MPa^{0.5}, and H₁=13.0 MPa^{0.5}, and
combining the reaction product and the one or more solvents to form a premix consisting of the reaction product and the one or more solvents.

7. The method of claim 6, wherein the one or more solvents comprises caprylic/capric triglyceride, isopropyl myristate, citronellyl acetate, acetone, triglycerides, an triglycerides-containing oil, or a combination thereof.

8. The method of claim 7, wherein at least some of the triglycerides are provided by a vegetable oil.

9. The method of claim 6, wherein the reaction product is present in an amount of 5-25% by weight of the cosmetic premix composition.

10. The method of claim 6, wherein the natural or food-derived oil is linseed oil.

11. The method of claim 6, further comprising combining one or more additional materials with the premix.

12. A cosmetic composition comprising:
a cosmetic premix consisting of:
a reaction product of a natural or food-derived oil and a methacrylate or acrylate polymer; and
one or more solvents, each solvent having Hansen Solubility Parameters that satisfy
$$Ra \leq \sqrt{4 {( D - {D}_{1} )}^{2} + {( P - {P}_{1} )}^{2} + {( H - {H}_{1} )}^{2}} ,$$
where Ra=13.4 MPa^{0.5}, D₁=16.8 MPa^{0.5}, P₁=4.8 MPa^{0.5}, and H₁=13.0 MPa^{0.5}; and
one or more additional materials.

13. The cosmetic composition of claim 12, wherein the one or more additional materials includes one or more additional solvents having Hansen Solubility Parameters satisfy
$$Ra > \sqrt{4 {( D - {D}_{1} )}^{2} + {( P - {P}_{1} )}^{2} + {( H - {H}_{1} )}^{2}} ,$$
where Ra=13.4 MPa^{0.5}, D₁=16.8 MPa^{0.5}, P₁=4.8 MPa^{0.5}, and H₁=13.0 MPa^{0.5}.

14. The cosmetic composition of claim 12, wherein the one or more additional materials includes one or more pigments, a filler, a polyol, a polyester of polyglycerol having 2 to 10 glycerol units, and/or a glyceryl ester of a fatty acid.
